(19) United States
(12) Patent Application Publication
(10) Pub. No.: US 2010/0331201 A1
(43) Pub. Date: Dec. 30, 2010

(54) PROTEIN BIOCHIP FOR THE DIFFERENTIAL SCREENING OF PROTEIN-PROTEIN INTERACTIONS

(75) Inventors: Stefan Müllner, Langenfeld (DE); Angelika Lüking, Bochum (DE); Verena Trappe, Dortmund (DE)

Correspondence Address: CONNOLLY BOVE LODGE & HUTZ, LLP, P O BOX 2207, WILMINGTON, DE, 19899, US

(73) Assignee: PROTAGEN Aktiengesellschaft, Dortmund (DE)

(21) Appl. No.: 12/304,060

(22) PCT Filed: Jun. 11, 2007
(86) PCT No.: PCT/DE2007/001029

§ 371 Date: Aug. 13, 2010

(30) Foreign Application Priority Data
Jun. 9, 2006 (DE) 10 2006 027 259.5

(51) Int. Cl. C40B 30/04; C40B 40/10; G01N 33/53

(52) U.S. Cl. 506/9; 506/18; 436/86

(57) ABSTRACT

The present invention relates to a system of protein binders containing at least one protein binder presented in at least one native form and at least one non-native form and its use including a method for discrimination and/or differential screening of suitable protein binders in native and non-native form.

The present invention relates to a system of protein binders containing at least one protein binder presented in at least one native or natural form and at least one non-native form and the use of the same, or methods of discrimination or differential screening of suitable protein binders which recognize the native or non-native form of protein binders. Increasingly, protein biochips are being used in industrial fields for analytical and diagnostic applications and in the development of pharmaceuticals.

In particular, major gains in information have been achieved by using protein biochips in analyzing the genome and gene expression. This has enabled the rapid and highly parallel detection of a multitude of specifically binding analysis molecules in a single experiment. To generate protein biochips, it is necessary for the required proteins to be available. Protein expression libraries have been developed for this purpose. One option involves the high throughput cloning of defined open reading frameworks (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is highly dependent upon the progress of genome sequencing projects and the annotation of these gene sequences. Moreover, the determination of the expressed sequence is not always clear due to differential splicing methods. This problem can be overcome by using cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). In this case, the cDNA of a specific tissue is cloned into a bacterial or yeast expression vector. The vectors used for expression are generally characterized in that they carry inducible promoters with which the time of protein expression can be controlled. Expression vectors also comprise sequences for so-called affinity epitopes or affinity proteins, which permit the specific detection of the recombinant fusion proteins by means of an antibody directed against the affinity epitope and enable specific purification through affinity chromatography (IMAC).

For example, the gene products of a cDNA expression library from human fetal brain tissue in the Escherichia coli bacterial expression system were arranged in high-density format on a membrane, and could be screened successfully using various antibodies. It was demonstrated that the ratio of full-length proteins is at least 66%. The recombinant proteins of this library were also successfully expressed and purified in high throughput (Braun P., Hu, Y., Shen, B., Halleck, A., Koundinya, M., Harlow, E. and LaBaer, J. (2002) Proteome-scale purification of human proteins from bacteria. Proc Natl Acad Sci U S A, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Such protein biochips based upon cDNA expression libraries are the particular subject of WO 99/57311 and WO 99/57312.

Antigen/antibody presenting systems are also described as protein biochips (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

For example, using protein biochips in individual experiments, the binding specificity of various monoclonal antibodies such as anti-HSP90β, anti-GAPDH and anti-α-tubulin were successfully analyzed on a protein microarray consisting of 96 human recombinantly expressed proteins (Lueking (1999)). Additionally, the cross-reactivity of two monoclonal antibodies against approximately 2500 different proteins have been studied (Lueking, A., Possling, A., Huber, O., Beveridge, A., Horn, M., Eickhoff, H., Schuchardt, J., Lehrach, H. and Cahill, D. J. (2003) A Nonredundant Human Protein Chip for Antibody Screening and Serum Profiling. Mol Cell Proteomics, 2, 1342-1349).

However, there is an urgent need to differentiate between non-native forms and native forms of protein binders and to identify these differences, which are generated, for example, based upon synthetic/recombinant production or the isolation/purification of modified protein structures.

For example, most recombinant proteins (for example, therapeutic proteins) have specific differences from the physiological or native homologues, whether this is in folding, for example, in the presence of auxiliary sequences, so-called tags, N-or C-terminal modifications, post-translational modifications, such as glycosylations, phosphorylations, acylations, alkylations, oxidations, etc., for example, or in the presentation of a binding site, which lead to changes in the protein/protein interaction (protein to be bound on a protein binder).

The invention therefore relates to the object of enabling a differential screening of protein binders for differentiating the native form from a non-native form, thereby allowing suitable native and non-native forms to be screened out.

The object is attained by providing a system of protein binders containing at least one protein binder presented in at least one native form and at least one non-native form, wherein each respective form has at least one recognition signal for binding one or more proteins, along with means of detecting the binding result.

The binding result permits a statement regarding the suitability or differentiation of the non-native form as compared with the native form of a protein binder.

The invention is to be used particularly advantageously for controlling and monitoring the quality of (non-native) proteins which are subject, for example, to any non-natural purification process or production method (such as a recombinant method), and whose homogeneity, bioequivalence, batch uniformity, activity and function as compared with the native form of the protein are to be determined.

Therefore, such a system of the invention is suitable for the differential screening or choice and selection of at least one native or non-native form.

According to the invention, the recognition signal is preferably an epitope and/or paratope.

Within the context of this invention, the native form of a protein binder is one whose function corresponds to that of the protein binder (e.g., immunoglobulins, therapeutic proteins, structural proteins, membrane proteins, etc.). In this, the original function is ensured by the sequence, conformation or configuration of the protein structure (primary, secondary, tertiary, quaternary structure). For example, the protein binder has an epitope (paratope), which in its native form can be definitely recognized by an antibody or a molecule which is similar in terms of binding affinity and selectivity (antigen). In particular, the native form is one which can be obtained or is known from a living organism (in vivo).

In addition, the native form can be an accepted standardized form of a protein, for example, for a specific function. Such a function is not necessarily a diagnostic or physiological function of a protein.

Within the context of this invention, the non-native form of a protein binder is one in which the function may be altered, even impaired, or modified as compared with the native protein binder. In this connection, the original function is not ensured by the sequence, conformation or configuration or modification of the altered or modified protein structure. For example, the protein binder has a modified epitope (paratope) which in its non-native form cannot be differentiated from its native form by an antibody or a molecule (antigen) that is similar in terms of binding affinity and selectivity.

A non-native form of a protein binder is particularly one in which the protein binder has been produced synthetically or recombinantly, or the native protein binder has been physically or chemically denatured, and differs from the native form particularly in its conformation and configuration. The folding of the protein binder in its non-native form as compared with its native form is of particular significance.

Particularly with a change in the conformation and configuration of a protein binder, namely to its non-native form, the recognition signal (e.g., epitope, paratope) for a protein to be bound can be disrupted/altered or even removed, so that the addressing of the protein to be bound is misdirected.

Therefore, in a further embodiment of the invention, the non-native form is the denatured form of a protein. According to the invention, this denatured form of a protein is compared with the non-denatured form (or: native form). The production of denatured forms requires denaturing agents, for example (urea, acids, bases, SDS, guanidine hydrochloride, salt, etc.), or physical denaturing, for example, high temperatures, γ rays, UV rays, lasers, etc.

In a further embodiment, the native form is an unmodified form, and the non-native form is a modified form. The modified forms have structural changes from the unmodified forms, including but not limited to a removal of phosphates with phosphorylated proteins, a removal of carbohydrates with glycolized proteins, a removal of lip(o)ids with lip(o)idized proteins, or a removal of post-translational structures in the protein. Additionally, the modifications can involve the native form being chemically altered in some way (e.g., phosphorylation, glycolization, lip(o)idization, derivatizations). The modification can further involve ligating the native form (unmodified form) with an additional protein to form a fusion protein (modified form).

In a further embodiment, the native form (unmodified form) is modified by splitting the native form into fragments. This can involve enzymatic or chemical or physical splitting, for example.

In a further preferred embodiment, the native form is a naturally occurring target or marker, for example, especially a biomarker, which are suitable for the diagnosis or treatment of diseases in humans and animals, and which will be comparatively studied against a denatured (non-native) form or modified form. Such denatured or modified forms for comparison can be produced, for example, through a purification process, isolation process.

In a preferred embodiment, the protein binder is an antigen (epitope) or antibody (paratope), especially a monoclonal or polyclonal antibody. Additionally, protein binders which contain parts of an antibody, such as Fab or Fc fragments, are preferred. Also included are Affibody® (Affibody, Sweden).

The term “protein binder” within the context of this invention means that in the presence of a protein binder, a protein to be bound comes into contact with the protein binder or binds to it, or at least interacts with it. In the broadest sense, the protein to be bound is addressed to the protein binder or the protein to be bound recognizes the protein binder, or the protein binder has the potential to interact with a protein (e.g., antigen (epitope)/antibody (paratope) interaction).

Protein binders can be proteins, peptides, modified proteins/peptides, recombinant proteins/peptides, antibodies or a molecule that is similar in terms of binding affinity and selectivity, or antigens, or other proteins which can be represented on a protein biochip according to the invention.

Suitable proteins for binding on a protein binder within the context of this invention include but are not limited to: proteins, peptides, modified proteins/peptides, recombinant proteins/peptides, antibodies or antigens, or other proteins, proteides.

The proteins to be bound can be present in purified form and mixed or even in a heterogeneous protein mixture, such as a lysate or digest (e.g., lysates of microorganisms or plants, tissue lysate, mammalian cell lysate). This reflects the quality of the binder in complex mixtures, such as occur, for example, in immunohistochemistry.

In particular, the invention relates to a system of one or more protein binders in which a first region has at least one native form of a protein binder and a second region has at least one non-native form of a protein binder, and these regions form a unit, wherein this unit is accessible to at least one protein to be bound, preferably at the same time under standardized conditions.

In a further embodiment, the protein binder can be represented in its respective form, native or non-native, in different quantities in the first and/or second region. This permits a variation in sensitivity. The first and second regions can each have the totality of protein binders, i.e., a sufficient number of different protein binders. These can also be present in a non-native or native form. At least 96 to 25,000 (numerical) or more different protein binders are preferable. However, more than 2,500, especially preferably 10,000 or more protein binders, resulting, for example, from an expression library are preferred.

The invention of such a system of protein binders therefore also relates to a diagnostic device or a protein biochip or protein microarray. Within the context of this invention, “system” is synonymous with “array” and to the extent that this “array” is used to identify proteins to be bound to protein binders, it is also understood to mean “assay.” In a preferred embodiment, the system is configured in such a way that the protein binders represented in the system are present in the form of a grid. Further, systems which permit a high-density array of protein binders are preferred. Such high-density systems are disclosed, for example, in WO 99/57311 and WO 99/57312.

The protein binders can be fixed, spotted, or immobilized in the system on a solid substrate.

In a further embodiment, the protein binders are present as clones. Such clones may be obtained, for example, by using a cDNA expression library according to the invention (Büssow et al. 1998 (supra)). In a preferred embodiment, such expression libraries containing clones are obtained using expression vectors from an expressed cDNA library. These expression vectors preferably contain inducible promoters. Induction of the expression may be carried out, for example, using an inducer, such as IPTG, for example. Suitable expression vectors are described in Terpe, et al. (Terpe T. Appl Microbiol Biotechnol. 2003 January; 60(5): 523-33).

Expression libraries are known to one skilled in the art; they can be produced according to standard sources, such as Sambrook et al, “Molecular Cloning, A laboratory handbook, 2^{nd} edition (1989), CSH press, Cold Spring Harbor, N.Y. Also preferred are expression libraries which are tissue-specific (e.g., human tissue, especially human organs). The invention further includes expression libraries that can be obtained by exon trapping. The term expression bank may be used as a synonym for expression library.

Also preferred are protein biochips or corresponding expression libraries which do not have any redundancy (so-called: Uniclone® library) and can be produced according to the teaching of WO 99/57311 and WO 99/57312, for example. These preferred Uniclone libraries have a high percentage of non-defective fully expressed proteins of a cDNA expression library.

Within the context of this invention, the clones can also be, but are not limited to, transformed bacteria, recombinant phages or transformed cells from mammals, insects, fungi, yeasts or plants.

The clones are fixed, spotted, or immobilized on a solid substrate.

Additionally, the protein binders in their respective forms can be present in the form of a fusion protein, which contains at least one affinity epitope, or “tag,” for example. The tag can be one such as those contained in c-myc, His-tag, Arg-tag, FLAG, alkaline phosphatase, V5-tag, T7-tag, or strep-tag, HAT-tag, NusA, S-tag, SBP-tag, thioredoxin, DsbA, a fusion protein, preferably a cellulose-binding domain, green fluorescing protein, maltose binding protein, calmodulin binding protein, glutathione S-transferase or lacZ.

In all embodiments, the term “solid substrate” encompasses embodiments such as a filter, a membrane, a magnetic bead, a silicon wafer, glass, metal, a chip, a mass spectrometry target or a matrix.

As a filter, PVDF, nitrocellulose or nylon is preferred (e.g., Hybond N+ Amersham).

In a further preferred embodiment of the system of the invention, said system corresponds to a grid having the dimensions of a microtiter plate (96 wells, 384 wells, or more), a silicon wafer, a chip, a mass spectrometry target or a matrix.

Once the protein to be bound has contacted a protein binder, evaluation is conducted using commercial image analysis software (GenePix Pro (Axon Laboratories), Aida (Raytest), ScanArray (Packard Bioscience)), for example.

The visualization of protein/protein interactions according to the invention (e.g., protein on protein binder, such as antigen/antibody) or “means of detecting the binding result” can be carried out through customary means, for example, fluorescence labeling, biotinylation, or radioisotope marking. For example, read-out can be conducted using a microarray laser scanner.

The invention further relates to a method of differential screening of suitable protein binders in non-native form, in which the detection of the binding result of a protein on a protein binder in non-native form is sufficient. Corresponding protein binders in non-native form are screened out.

The invention therefore relates to a method of the differential screening or discrimination of protein binders which have or recognize at least one native form and at least one non-native form, wherein the respective form has at least one recognition signal for binding one or more proteins, involving contacting the native form and non-native form with at least one binding protein, and detection of the binding result. A binding result is present when there is a positive detection of the binding protein in a recognition signal of the protein binder, particularly when both the native form and the non-native form of the protein binder have a binding result from the protein to be bound.

In a further embodiment of the invention, the method of the invention particularly advantageously allows a discrimination of such protein binders with an epitope, paratope, in which a comparative study of the native form with the non-native form is conducted. In this case, it is important whether the respective epitope, paratope sequence is relevant to the protein to be bound, or whether non-epitope, non-paratope sequences of the non-native form are relevant.

The invention further relates to a method of identifying and characterizing protein binders which present at least one native form and at least one non-native form, wherein the respective form has at least one recognition signal for binding one or more proteins, involving a contacting of the native form and native form with at least one binding protein, and detection of the binding result. A binding result is present when there is positive detection of the binding protein in a recognition signal of the protein binder, particularly when both the native form and the non-native form of the protein binder have a binding result from the protein to be bound.

The method of the invention is carried out using the above-described embodiments, particularly systems, in particular, the native form is an unmodified or non-denatured form and the non-native form for comparison is a denatured or modified form.

The methods of the invention permit such non-native forms which have a binding result to be reliably screened out.

The methods of the invention for screening suitable epitopes/paratopes are preferably used.

The invention therefore also relates to the use of a system according to the invention for the differential screening of suitable protein binders for differentiating the non-native form from the native form.

The differential screening particularly permits the selection or discrimination of such protein binders in non-native form which therefore do or do not have a homologous function in terms of protein/protein interactions.

### EXAMPLE AND FIGURES

This example is intended solely to elucidate the invention, without limiting the invention to this example.

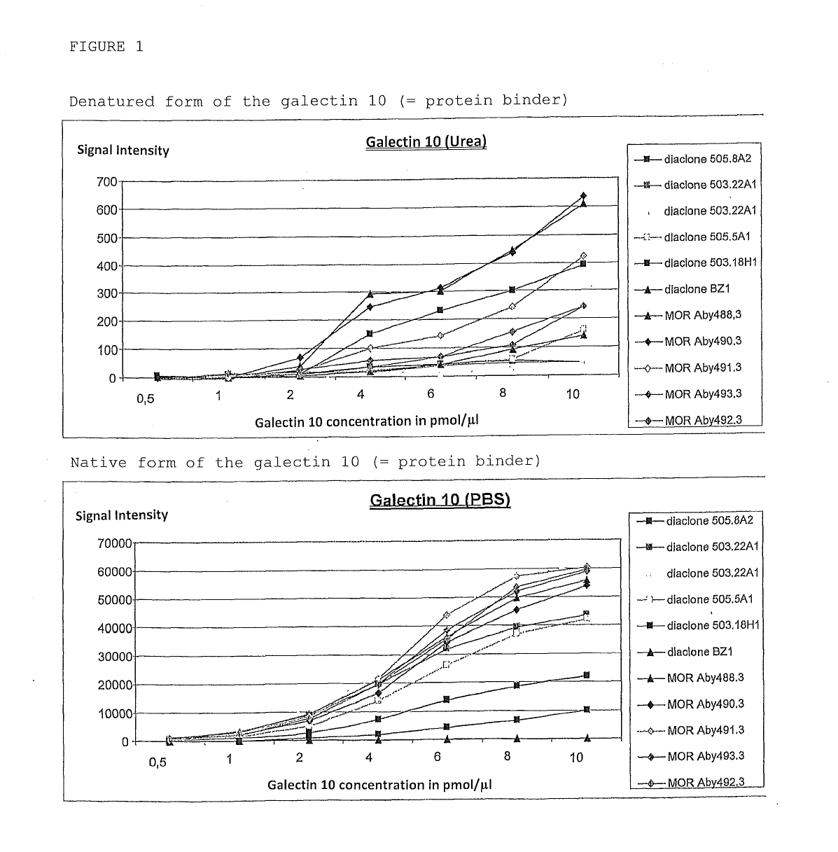

In FIG. 1, the differential screening of the protein binder galectin 10 in native form (non-denatured) and non-native form (denatured (denaturing agent: urea)) is shown with different antibodies to be bound. Based upon the binding curves (signal intensities vs. concentrations/spot), the comparative binding result can be determined.

For example, Diaclone 503.18H1 recognizes the native form of galectin 10 less effectively than the other antibodies, and the denatured form of the galectin 10 more effectively.

The antibody MOR Aby491.3 best recognizes the native form of galectin 10, but recognizes the denatured form of the galectin 10 much less effectively than the other antibodies.

1. System of protein binders containing at least one protein binder presented in at least one native form and at least one non-native form, wherein the respective form has at least one recognition signal for binding one or more proteins, along with means of detecting the binding result.

2. System of protein binders according to claim 1, characterized in that the native form is an unmodified or non-denatured form and the non-native form is a denatured or modified form.

3. System of protein binders according to claim 1, characterized in that the non-native form is produced recombinantly or synthetically.

4. System of protein binders according to claim 1, characterized in that the modified form has structural modifications in relation to the unmodified form, or the modified form is chemically altered in relation to the unmodified form.

5. System of protein binders according to claim 1, characterized in that the native form is a target or marker.

6. System of protein binders according claim 1, characterized in that the protein binders are proteins, peptides, modified proteins/peptides, recombinant proteins/peptides, preferably antigen and/or antibodies or a molecule that is similar in terms of binding affinity and selectivity.

7. System of protein binders according to claim 1, characterized in that the recognition signal is an epitope or paratope.

8. System of protein binders according to claim 1, characterized in that the system has a first region of a protein binder in native form and has a second region of a protein binder in non-native form, and these regions form a unit, wherein this unit is accessible to at least one protein to be bound.

9. System of protein binders according to claim 1, characterized in that the first and/or second region contains a total of at least 96 to 25,000 or more protein binders arranged in the form of a grid, particularly having the dimensions of a microtiter plate, a silicon wafer, a chip, a mass spectrometry target or a matrix.

10. System according to claim 1, characterized in that the protein binders are clones of a cDNA expression library, particularly transformed bacteria, recombinant phages or transformed cells of mammals, insects, fungi, yeasts, or plants.

11. System according to claim 1, characterized in that the protein binders are present as fusion proteins, particularly an affinity epitope or a tag, such as is contained in c-myc, His-tag, Arg-tag, FLAG, alkaline phosphatase, V5-tag, T7-tag or strep-tag, HAT-tag, NusA, S-tag, SBP-tag, thioredoxin, DsbA, a fusion protein, preferably a cellulose binding domain, green fluorescing protein, maltose binding protein, calmodulin binding protein, glutathione S-transferase or lacZ.

12. System of claim 1, characterized in that the proteins to be bound are present in purified form and mixed or in a heterogeneous protein mixture, such as a lysate or digest.

13. Protein biochip or protein microarray comprised of a system according to claim 1.

14. (canceled)

15. Method of the differential screening or discrimination of protein binders which have at least one native form and at least one non-native form,
wherein the respective form has at least one recognition signal for binding one or more proteins,
contacting the native form and the non-native form with at least one binding protein and detecting the binding result.

16. Method of identifying and characterizing protein binders which present at least one native form and at least one non-native form, wherein the respective form has at least one recognition signal for binding one or more proteins,
contacting the native form and the non-native form with at least one binding protein and detecting the binding result.

17. Method according to claim 15, wherein the native form is an unmodified or non-denatured form, and the non-native form is a denatured or modified form.

18. Method according to claim 15, characterized in that the method is carried out on a system of protein binders containing at least one protein binder presented in at least one native form and at least one non-native form, wherein the respective form has at least one recognition signal for binding one or more proteins, along with means of detecting the binding result.

19. Method according to claim 15, characterized in that non-native forms are screened out using a binding result.
